(19) United States
(12) Patent Application Publication
(10) Pub. No.: US 2009/0305270 A1
(43) Pub. Date: Dec. 10, 2009

(54) ANTIGENIC SURFACE STRUCTURE OF SPERM CELLS ASSOCIATED WITH THE Y CHROMOSOME

(75) Inventors: Sophie Duban-Deweer, Villeneuve-d'Ascq (FR); Luc Liegeois, Raimbeaucourt (FR)

Correspondence Address: HARNESS, DICKEY & PIERCE, P.L.C., P.O. BOX 828, BLOOMFIELD HILLS, MI, 48303, US

(73) Assignee: Genes Diffusion, Douai (FR)

(21) Appl. No.: 12/306,154

(22) PCT Filed: Jun. 21, 2007
(86) PCT No.: PCT/FR2007/001026

§ 371 Date: Feb. 5, 2009

(30) Foreign Application Priority Data
Jun. 22, 2006 (EP) 06370021.5

(51) Int. Cl. C12Q 1/68; C07K 14/47; C07K 16/18

(52) U.S. Cl. 435/6; 530/350; 530/387.9

(57) ABSTRACT

The present invention relates to an antigenic surface structure of sperm cells associated with the Y chromosome, to the molecules, in particular antibodies, directed against this antigenic structure and to a method for characterizing cells carrying only the Y chromosome through the interaction between this antigenic structure and the molecules directed against said structure.

### FIELD OF THE INVENTION

The present invention relates to an antigenic structure present on sperm cells (spermatozoa) and associated with the Y chromosome and molecules, in particular antibodies or portions of the latter, directed against this antigenic structure.

The present invention also relates to a method enabling the presence of the Y chromosome in a cell to be characterised, in particular in a sperm cell (spermatozoon).

The invention also relates to a method enabling the sex of the progeny of mammals, including humans, to be characterised by a separation of the spermatozoon cells determined to be male and carrying the Y chromosome, and based on the use of the interaction of this antigenic structure with preferably labelled molecules, in particular labelled antibodies, directed against this antigenic structure.

### TECHNICAL BACKGROUND AND PRIOR ART UNDERLYING THE INVENTION

In natural cross-breeding and artificial insemination (AI) conditions, the gender ratio is approximately 50% male (XY) and 50% female (XX).

Farmers and other animal breeders have for a long time expressed their desire to increase the probability of producing a progeny of selected sexes (preferably through the selection of spermatozoon cells carrying the Y chromosome or carrying the X chromosome prior to an insemination of the mammals).

Beyond the obvious physiological aspects, selecting progeny in function of gender has significant economic consequences when taking these applications into consideration for meat production (for goats, cattle and swine), but also when considering these applications for competitions involving animals (in particular horses and dogs).

Different technological approaches have been taken in order to achieve the selection of gender in mammals, both for the interest in the animal science of species and for species facing extinction as well as for domestic animals and assisted reproduction in humans. There are two alternatives for this: the separation of the sperm cells carrying the X chromosome from those carrying the Y chromosome or the determination of the sex of an embryo prior to reimplantation.

In humans, the selection of the sex of an embryo implies strong ethical constraints that could be eliminated through sexing. Sexing of sperm cells could also have clinical applications in order to prevent diseases linked to the X chromosome in the human species, such as Duchenne muscular dystrophy, haemophilia A or B, retinal depigmentation, Lesch-Nyhan syndrome or Fragile X syndrome, leading to mental retardation (ZARUTSKIE et al. 1989). There are about 6000 genetic anomalies and more than 370 of them are linked to the X chromosome (COTINOT et al. 1993, JOHNSON et al. 1993).

Various attempts have been made to be able to select progeny according to sex. Those that appear the most interesting in terms of achieving the desired result have been directed towards the separation and selection of X or Y sperm cells.

Any separation technique, in order to be efficient, must use phenotypic differences between the X and Y sperm cells. The separation of the spermatozoa carrying the X chromosome from those carrying the Y chromosome is linked to the detection of at least one difference between these phenotypes. In this context, separation trials have been carried out on the basis of visible various differences (chemical or physical) between the spermatozoon cells X and Y (such as a difference in fluorescence, size, mass, density, swimming speed, sensitivity to pH, adhesion to Sephadex, etc).

Means for the immunological separation of the spermatozoa carrying the X chromosome from the spermatozoa carrying the X chromosome have also been suggested.

U.S. Pat. No. 4,448,767 describes a method that requires the development of antibodies against the markers of the specific cell surface capable of discriminating between the X cells and the Y cells.

An H-Y antigen has been used as a marker for the separation of sperm cells carrying the X chromosome or the Y chromosome (Ali et al. 1990; Peter et al. 1993). Nevertheless, other researchers have reported their inability to isolate cells of the X or the Y type by using the H-Y antigenic marker (Hendriksen et al. 1993; Sins et al. 1998). The current consensus is that the H-Y antigen does not allow a separation between cells carrying the X chromosome or the Y chromosome.

U.S. Pat. No. 5,660,997 describes the use of membranous proteins linked to sex (SAM proteins). These SAM proteins are characterised by a particular molecular weight determined on SDS polyacrylamide gel (PAGE) and by an isoelectric point (PI) determined by an immobilisation on a gel with a pH gradient (IPG). In chincilla dogs, the SAM-X proteins have molecular weights of respectively 19 kDa, 25 kDa, 29 kDa, 32 kDa, 39 kDa, 72 kDa and 120 kDa, while the SAM proteins of the Y chromosome have molecular weights of 15 kDa, 45 kDa, 55 kDa, 64 kDa and 125 kDa.

Nevertheless, this patent application does not give any data on efficiency of the method or any proof that these characterised proteins are in effect linked to the X or Y chromosome of the cells and that said link has as a consequence the significant deviation of the sex ratio in mammal species.

U.S. Pat. No. 5,021,244 describes an antibody that fixes itself in a specific manner to the membranes of the cells carrying the X chromosome (X-SAM). Said antibody (preferably monoclonal) is essentially deprived of antibodies capable of fixing membranous proteins associated with the Y chromosome or associated with H-Y antigens, or with non-specific sex components present in the plasma membrane of the cell.

Patent application CA 1341328 describes membranous proteins associated with sex, one protein of which has a molecular weight of 9.6 kDa and an isoelectric point of 6.58.

### OBJECTS OF THE INVENTION

The present invention aims to provide a new antigenic surface structure of sperm cells associated with the Y chromosome and enabling a discrimination to be obtained between the sperm cells (spermatozoa) carrying the Y chromosome and cells carrying the X chromosome.

The present invention also aims to provide preferably labelled molecules, in particular labelled antibodies, such as monoclonal antibodies or portions of the latter, directed specifically against this antigenic structure and to use the interaction between this antigenic structure and these molecules to obtain a specific labelling of these cells, and also in order to be able to discriminate and possibly separate cells carrying only the Y chromosome from the other cells (carrying the X chromosome and/or possibly carrying the Y chromosome).

The present invention also relates to a method of inseminating mammals using said discriminated cells.

### SUMMARY OF THE INVENTION

The present invention relates to an antigenic surface structure of sperm cells (spermatozoa) specific to the Y chromosome and having a molar mass inferior to 15 kDa, preferably inferior to 10 kDa, more particularly presenting a molar mass comprised between 5 and 10 kDa, and an isoelectric point superior to 9, preferably comprised between approximately 9 and approximately 11, more particularly comprised between approximately 9 and approximately 10. Preferably, the antigenic structure presents a molar mass of respectively approximately 5 kDa and a PI of approximately 9.35, or approximately 7 kDa and a PI of approximately 9.07 or of approximately 10 kDa and a PI superior or equal to 10.

The specificity of this antigenic structure can be determined by a Western Blot analysis, which labels specifically and exclusively this relevant antigenic structure present only on the Y chromosome.

Consequently, a first object of the invention relates to the use of the relevant antigenic structure of the invention for the specific labelling of cells expressing the Y chromosome as opposed to cells not expressing this Y chromosome (cells carrying only the X chromosome).

This analysis can be carried out on live cells through a labelling of the surface of the cell or on cellular extracts, for instance by means of a Western Blot analysis.

A further aspect of the present invention relates to molecules, preferably labelled and directed against (and specific to) this antigenic structure.

These molecules are preferably labelled antibodies or hypervariable and specific portions of these antibodies directed (specifically) against this antigenic structure. These molecules can also be nanobodies™ which are a new class of therapeutic proteins derived from antibodies. These examples of molecules are derived from antibodies from the camelid family which are deprived of the specific light chain of antibodies but which maintain the complete capacity to fix (specifically) antigens. It is thus possible to obtain nanobodies constituted exclusively of the variable domain portion of the extremity of an antibody (www.alynx.com).

Preferably, the antibody is a monoclonal antibody.

A further aspect of the present invention concerns the cell (hybridoma) capable of producing these antibodies or the hypervariable and specific portions of these antibodies.

The present invention also relates to a diagnostic kit (for sexing) comprising said molecules (possibly labelled) directed specifically against this antigenic structure, in particular said antibodies or the hypervariable portions of the latter, as well as all the media and reagents intended to characterise the interaction between these antigenic structures and said molecules directed (specifically) against them.

These media and reagents are for instance wash solutions and labelling elements of said molecules directed against (specific to) the antigenic structure (such as calorimetric, luminescent, chemoluminescent, bioluminescent, fluorescent or radioactive markers). These media or reagents can also include solid supports capable of interacting with said molecules directed against (specific to) the antigenic structure. Solid supports suitable for interacting with these molecules are beads, agarose beads or metallic beads (in particular magnetic beads), of an adequate size (between approximately 50 nm and approximately 4 or 9 μm) so as to be able to efficiently fix and label living cells and subsequently to obtain a separation of sperm cells specifically carrying the Y chromosome from a mix of sperm cells.

The kit according to the invention can also include means for certifying the sexual characteristics of the chromosomes carried by said discriminated spermatozoa. These means are constituted by a gene amplification kit of specific genes of either the X chromosome or the Y chromosome. These means are preferably constituted by initiators, enzymes and reaction media enabling a gene amplification by a method chosen from the group constituted by PCR, LCR or NASBA.

The kit according to the invention can also be used to confirm if other means of separating the cells (via physical, chemical or biotechnological methods) are efficient. The kit according to the invention can thus be used as a means of certifying the presence of the Y chromosome on the cells or in cellular extracts.

A further aspect of the present invention concerns a solid support comprising said molecules directed specifically against the antigenic structure of the invention, said molecules being fixed on a solid support surface, in a direct or indirect manner (via an interaction by a couple of molecules (a linked pair) such as biotins and streptavidins/(or) avidins) and capable of specifically fixing sperm cells carrying the Y chromosome, via the interaction between the antigenic structure and said molecules.

The solid support can, for instance, consist of the solid support of a chromatography column, multiple-well plates, beads (of which the size is preferably comprised between approximately 50 nm and approximately 4 μm or approximately 9 μm, preferably between approximately 100 and approximately 400 or approximately 500 nanometres), in particular magnetic beads, enabling an interaction between the antigenic structure of the cells and the molecules directed against (specific to) these, enabling a discrimination and, advantageously, enabling a separation of the different categories of cells (separation of the cells carrying only the Y chromosome from the other cells (cells carrying the X chromosome and possibly other cells carrying the Y chromosome)).

The present invention also relates to a method of discrimination (non-therapeutic) and possibly of separation between the cells carrying the Y chromosome or the X chromosome through the use of the interaction between this antigenic structure and these molecules, in particular these antibodies or portions of the latter directed against (specific to) this antigenic structure.

The method of the invention comprises the following steps:
placing the sperm cells in contact with the molecules or the solid support of the invention (comprising said molecules) directed (specifically) against the antigenic structure of the invention, which is specific to the Y chromosome and presents at the surface of said cells, in conditions suitable for obtaining a specific interaction (linking or labelling) between said antigenic structure and said molecules;
linking or labelling of cells carrying the Y chromosome by the interaction of said molecules with said antigenic structure, said labelling being obtained by a signal resulting from the interaction between said antigenic structure present at the surface of the cells and said molecules directed against these;
possibly separating the cells linked or labelled by said molecules from the cells not linked and/or labelled by said molecules;
possibly collecting the linked or labelled cells or the other cells (non-linked and non-labelled and mostly carrying the X chromosome);
possibly one or several inseminations (non therapeutic) of a mammal by said cells (insemination of a mammal by said collected cells carrying only the Y chromosome) as well as possibly one or several inseminations (non therapeutic) of mammals by the other collected cells carrying mostly the X chromosome.

According to the invention, the molecules directed against (specific to) the antigenic structure are antibodies (preferably monoclonal) or a hypervariable portion of these. These antibodies can be labelled directly or indirectly by a calorimetric, luminescent, chemoluminescent, fluorescent or radioactive labelling element. This labelling can be a direct or indirect labelling.

Direct labelling involves a covalent fixation of the marker (colorimetric, luminescent, fluorescent, radioactive) on said molecule, preferably on said antibody.

Indirect labelling involves linking the first molecule with a second molecule already linked to this marker. For instance, the first molecule (being an antibody) can be made to react with a second molecule (a second antibody) directed (specifically) against the constant region of the first antibody, the second antibody preferably being linked in a covalent manner to the calorimetric, luminescent, chemoluminescent, fluorescent or radioactive marker, an enzyme capable of producing this marker or a solid support (beads).

In the method according to the invention, the characterisation of the cells is obtained by placing the molecules of the invention in contact with the cells. This operation is carried out in media enabling the survival of said cells, but also enabling the elimination of any interference with components susceptible of interfering with the interaction between said cells and said molecules directed against (specific to) the antigenic structure of the cells.

The separation and collection step of the cells carrying only the Y chromosome and the other cells is carried out as a result of the labelling of the cells by the molecules interacting with the antigenic structure. This separation can preferably take place on a solid support (preferably magnetic beads) on which said molecules have been fixed and with which the cells are likely to interact.

A medium allowing the elution of the other non-linked or non-labelled cells enables an efficient separation to be achieved. This separation and possibly collection step of the linked or labelled cells is known to the person skilled in the art.

The step involving the insemination of mammals by cells carrying only the Y chromosome and/or possibly of mammals by sperm comprising mostly cells carrying only the X chromosome (and possibly a weaker proportion of cells carrying the Y chromosome) is also carried out by methods well known to the person skilled in the art.

The term “sperm comprising mostly cells carrying only the X chromosome” indicates the fact that a proportion of cells superior to approximately 50%, preferably superior to approximately 75%, more particularly superior to approximately 90% or approximately 95%, carry only the X chromosome.

Via these insemination steps, it is possible to obtain exclusively male progeny or essentially, preferably exclusively, female progeny.

The mammals to be inseminated are preferably mammals of importance as meat-producing animals, such as goats, cattle and swine (preferably cattle), but can also include domestic animals, such as horses, rabbits, dogs or cats or mammals belonging to species facing extinction. The mammals can also be primates, including humans, in particular those whose parents carry hereditary diseases linked to the X chromosome, such as Duchenne muscular dystrophy, haemophilia A or B, retinal depigmentation, Lesch-Nyhan syndrome or Fragile X syndrome, leading to mental retardation.

The method of the invention can also possibly be combined with other procedures for separating cells carrying the Y chromosome or the X chromosome, for instance methods of separation based on chemical or physical differences associated with the cells carrying the X chromosome or with cells carrying the Y chromosome, such as differences of fluorescence, size, mass, density, swimming speed, sensitivity to pH, adhesion to Sephadex, etc.

The different stages of the method of the invention can be possibly combined with a step of specific characterisation of the sex of the cells carried out on the totality of selected cells or on a sample of the latter, in such a manner as to ensure that these characterisation steps of the cells are carried out in an optimal manner.

This characterisation procedure can for instance consist of one step or several steps of gene amplification (by PCR, LCR, NASBA, . . . ) or of one or several steps of adding one or several markers specific to the X chromosome or the Y chromosome to the cells.

According to the invention, the molecules directed against the antigenic structure (specific to the antigenic structure) can also be molecules capable of destroying said cells, for instance lytic antibodies or molecules including markers capable of being recognised in a specific manner and enabling a destruction by adequate means (laser) of the labelled cells carrying said antigenic surface structure, i.e. sperm cells associated with the Y chromosome.

Consequently, this method can be modified by obtaining the separation between the cells linked or labelled by said molecules, from the other cells, which are non-linked and non-labelled by said molecules, by means of a destruction of the labelled cells via the molecule directed specifically against said antigenic structure of the invention or via this means (laser) enabling only the labelled cells to be eliminated in a specific manner. This method will then include a collection of the non-linked and non-labelled cells mostly carrying the X chromosome and possibly an insemination of a mammal with only non-linked and non-labelled cells.

The present invention shall be described in more detail in the embodiment shown below, given by way of a non-limiting illustration of the method of the invention with reference to the attached drawings.

### BRIEF DESCRIPTION OF DRAWINGS

FIG. 1 shows the analysis of the total proteome of the cells enabling a protein fraction (fraction F) specific to the cells of the Y type to be isolated.

FIG. 2 shows the three isoforms of this protein fraction characterised by their molecular weight on an electrophoresis gel.

FIG. 3 shows the in vivo immunolocalisation labellings of sperm cells carried out with the antibody of the invention. The labelled and non-labelled cells are shown in FIG. 3.

### DETAILED DESCRIPTION OF INVENTION

### 1. Obtaining a Specific Protein Fraction

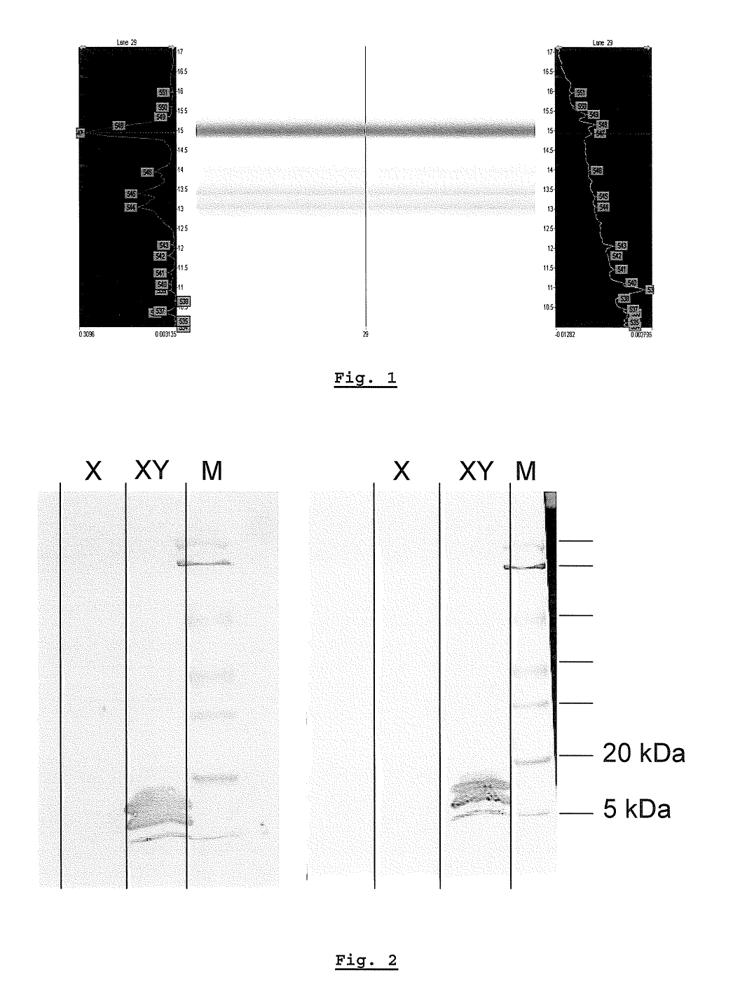

The analysis of the total proteome of the spermatozoa harvested for instance from a bovine animal is followed, with the help of an artificial vagina, by their dilution and cryopreservation treatment in PUC straws, which has enabled a protein fraction (Fraction F) to be isolated that is being specific to type Y cells (FIG. 1).

This protein fraction contains three isoforms:
molar masses respectively of 5, 7 and 10 kDa

### 2. Obtaining of Antibodies

The identified fraction was injected into two rabbits to obtain antibodies.

### 3. Verification of the Specificity of the Antibodies

The rabbit serum containing the polyclonal antibodies was tested for its specificity: Western Blot analyses clearly demonstrated that the polyclonal antibodies are specific for the sperm cells carrying the Y chromosome since only the protein fraction extracted from the male and female cells at 50/50 reacts with the serum.

The procedure was carried out according to the indications of the supplier of the “alkaline phosphatise conjugate substrate kit” 17016432 (BIO-RAD) by a dilution of the primary antibody at a ratio of 1:500.

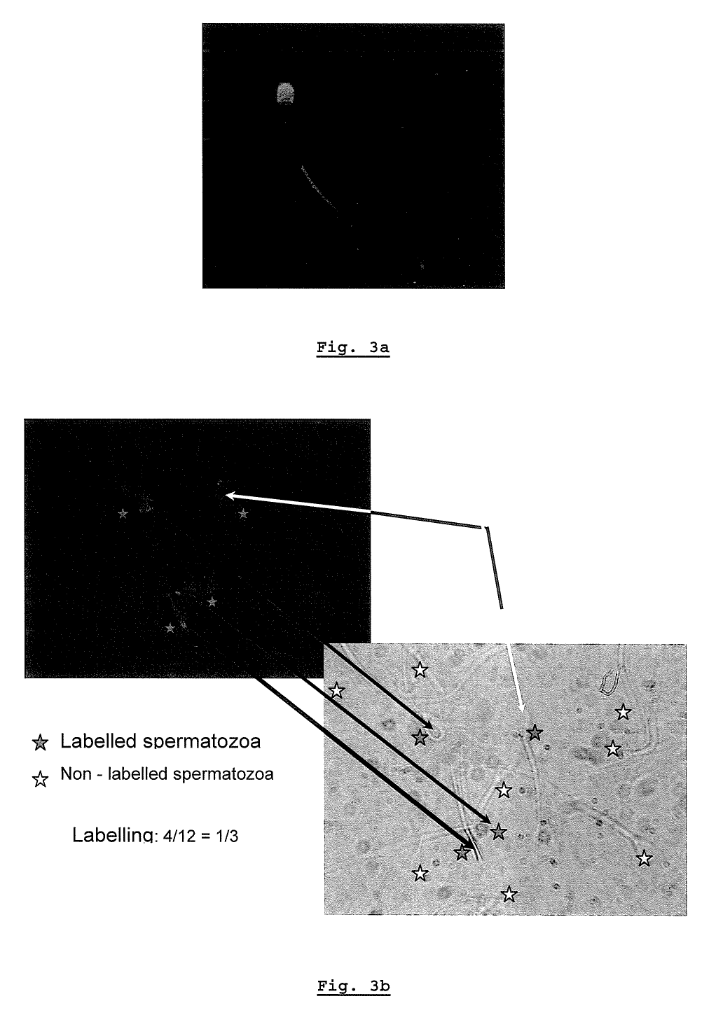

Protein extracts of non-sexed and sexed X cells are deposited on an acrylamide electrophoresis gel at 12.5%; following the transfer onto a PVDF membrane of these cellular extracts, the membrane is saturated overnight at 4° C. with the help of a TBST solution (tris-buffered saline containing 0.15% of Tween-20). Following 3 TBST washings, the membrane is placed in the presence of the rabbit serum (obtained by injection of the relevant chromatographic fraction) tested at suitable dilution (i.e. 1/500) by TBST during 1.5 hours. Following three new washings with TBST, the “rabbit anti-immunoglobin” goat antibodies (for instance Biorad product 170-6460) labelled with alkaline phosphatase are added over a period of two hours, after having been diluted according to the procedure of the supplier in the TBST buffer, three new washings are carried out, and then a last one in TBS without Tween-20 and phosphate 5-bromo, -4-chloro, -3-indoyle and nitroblue-tetrazolium are added according to the supplier indications (170-6432 of Biorad) until the result is obtained. A positive result corresponds to a precipitation of the substrate onto the membrane at the position of the relevant protein.
FIG. 2 shows 3 protein bands, which appear only in the extract containing cells carrying a mix of the X and Y chromosome, while no band appears in the protein extract containing the cells carrying only the X chromosome.

### 4. Verification of the Cellular Localisation of the Protein: Immunolocalisation Procedure on Cell Sperms

The biological material is constituted by cells that are harvested and used in the living state. 500 μl of fresh sperm are diluted in 4 ml of diluent set at 37° C. The sperm is slowly brought to an ambient temperature before being set at 4° C.

### 5. Immunolocalisation on Living Cells (in Eppendorf Pipette)

take 300 μl of diluted sperm and place in sterile Eppendorf pipette;
add primary antibody at 1:50 final;
incubate from 4 hours to one night at 4° C., shake from time to time;
centrifuge for 2 minutes at 600 g;
dispose of supernatant;
put 1 ml of diluent to rinse and suspend cells;
centrifuge for 2 minutes at 600 g;
dispose of supernatant;
re-suspend cells in 500 μl of diluent;
add secondary antibody at 1:50 final;
incubate for 1 to 2 hours at ambient temperature or overnight at 4° C.;
centrifuge for 2 minutes at 600 g;
dispose of supernatant;
add 1 ml of diluent for rinsing;
centrifuge for 2 minutes at 600 g;
dispose of supernatant;
re-suspend the pellet in 300 ml;
deposit 20 μl on a slide;
set up the slide;
observe the fluorescence under the microscope.

Not all of the cells of a sperm sample containing type X and type Y cells at a proportion of 50/50 are marked. It appears as though the distribution of the labelling is done at a rate of 35 to 50% of cells labelled by the antibody.

Following treatment enabling the attachment of antibodies, the cells are still living whether they have been fixed, labelled or not by the antibody.

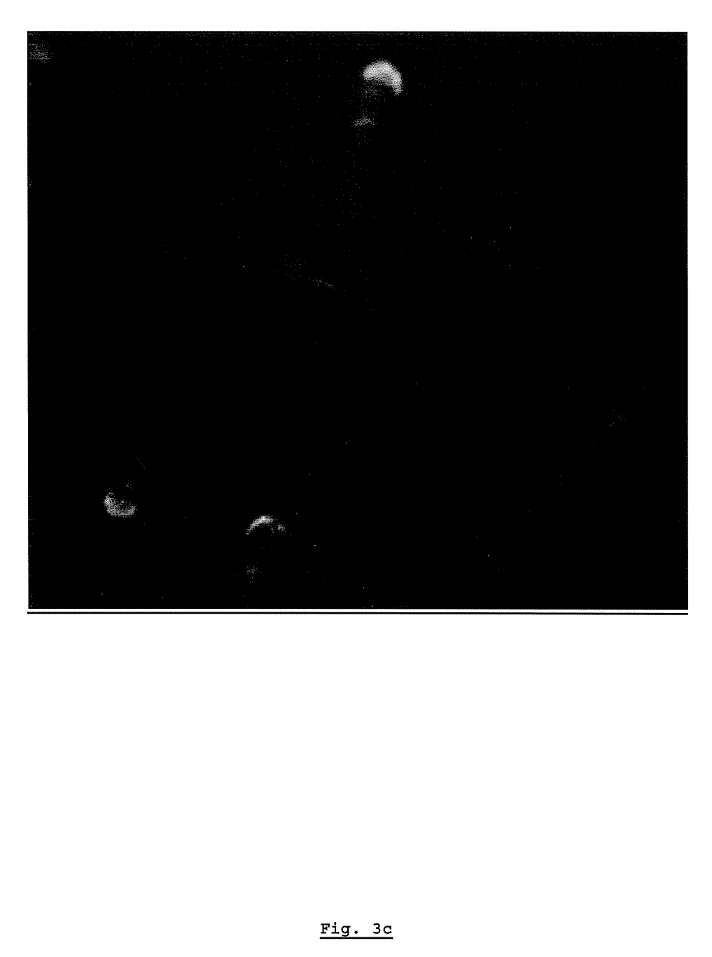

The antibody of the invention is thus specific to the surface of the cells of the Y type (FIG. 3). In FIG. 3, the labelling appears on the grey part of the drawings, the white part representing the cell.

### 6. Realisation of Cellular Subsets

The antibodies of the serum have been purified in order to eliminate the proteins of the rabbit serum, in particular albumen.

### PROCEDURE According to the Indications of the Supplier of the PURE-1A “Protein A Antibody Purification Kit” (SIGMA-ALDRICH)

Magnetic beads have been fixed on the purified antibodies. These antibodies fixed to the beads have been used to prepare subsets of sperm cells on the basis of the specificity of the antibody for a surface protein of spermatozoa carrying the Y chromosome. The procedure has been carried out according to the indications of the supplier of the DYNAL, dynabeads M450 epoxy kit.

### 7. Generation Procedure of the Two Subsets

The cells are used as quickly as possible post-harvest. They are diluted in a diluent (at a rate of “20” million cells per ml) enabling the integrity of the cells to be maintained while also enabling the attachment of the antibodies. The cells are placed into contact during 30 minutes with the antibodies, either directly coupled (grafted) to the magnetic beads (size comprised between approximately 50 and approximately 500 nm or approximately 9 μm, preferably between approximately 100 and approximately 400 or 500 nm), or indirectly coupled to these beads by the intermediary of a second antibody (goat anti-rabbit antibodies) that will fix antibodies contained in the serum, this placing into contact continues for 12 at a temperature comprised between approximately 4° C. and approximately 25° C. with gentle agitation and at a temperature comprised between approximately 4° C. and approximately 25° C. The cells attached to the antibodies armed with a magnetic bead are then submitted to a magnetic field in order to retain the cells of the Y type. The supernatant containing the X cells is recovered. The sperm cells of the supernatant are then ready to be used for artificial insemination.

### 8. Verification of the Sex of the Two Characterised Cellular Subsets

A verification test of the proportions of cells of each sex in the two cellular sets (fixed and non-fixed on the beads) is carried out. It consists in the amplification of a specific gene of either the X chromosome or the Y chromosome. This verification test of the constitution of the two sets enables the proportions of each of the cell types in the two sets to be compared.

Theory has it that, from the observations carried out, the retained set, supposedly that of the type Y cells, contains only cells carrying the Y chromosome. However, the cellular set contained in the supernatant, supposedly the type X cells, is represented mostly by type X cells, but contains a certain number of cells carrying the X chromosome. In effect it appears as though, from the in situ immunolocalisation observations, that all the spermatozoa of the type Y do not carry the antigen at their surface. The observations show that the relevant structure is present in the region of the head and of the second half of the flagella of the spermatozoon.

The cellular set contained in the supernatant has been used for artificial insemination. The sexing of the obtained embryos shows the following results in terms of sex ratio: (in progress).

9. Method for Inseminating Cattle with Spermatozoa

The cells selected by the method of the invention are kept in thin-walled PUC straws in liquid nitrogen at −196° C.

The straws are introduced into a gun (single-use metal tube covered in plastic) containing a piston suitable for ejecting the contained liquid into straws. By hand manipulation, the gun enables cells to be injected into the cervix at the base of the two uterine horns.

1-11. (canceled)

12. An antigenic surface structure of sperm cells associated with the Y chromosome having a molecular weight comprised between 5 kDa and 12 kDa and an isoelectric point superior to 9.

13. The antigenic structure according to claim 12, having an isoelectric point that is comprised between 9 and 11.

14. The antigenic structure according to claim 12, having a molecular weight of 5 kDa and an isoelectric point of 9.35, a molecular weight of 7 kDa and an isoelectric point of 9.7, or a molecular weight of 10 kDa and an isoelectric point superior or equal to 10.

15. A molecule specifically directed against the antigenic structure according to claim 12.

16. A molecule specifically directed against the antigenic structure according to claim 13.

17. A molecule specifically directed against the antigenic structure according to claim 14.

18. The molecule according to claim 15, having an antibody or a hypervariable portion of an antibody.

19. The molecule according to claim 16, having an antibody or a hypervariable portion of an antibody.

20. The molecule according to claim 17, having an antibody or a hypervariable portion of an antibody.

21. The molecule according to claim 18, having a monoclonal antibody.

22. The molecule according to claim 19, having a monoclonal antibody.

23. The molecule according to claim 20, having a monoclonal antibody.

24. A sexing kit comprising the molecule according to claim 15.

25. A solid support comprising the molecule according to claim 15, fixed on the surface of said solid support.

26. The solid support according to claim 25, and comprising beads, preferably magnetic beads.

27. The solid support according to claim 25, comprising beads possessing a diameter comprised between 50 nm and 9 μm, preferably comprised between 100 nm and 400 nm.

28. A method for discriminating between sperm cells carrying a Y chromosome and sperm cells carrying a X chromosome, said method comprising the following steps:
placing said cells in contact with a molecule specifically directed against an antigenic structure or a solid support comprising said molecule fixed on a surface of said solid support, under conditions suitable for obtaining a link between said molecules or said solid support and the antigenic structure;
linking or labelling of cells carrying the Y chromosomes by interaction of said molecules with said antigenic structure;
separating the cells labelled or linked by said molecules from the cells not labelled or linked by said molecules; and
collecting the labelled or linked cells or the non-labelled and non-linked cells.
